(19) United States
(12) Patent Application Publication
(10) Pub. No.: US 2004/0002675 A1
(43) Pub. Date: Jan. 1, 2004

(54) Flexible barrier film for a backing material for medical use

(75) Inventors: Jens Nierle, Hamburg (DE); Andreas Schabert, Seevetal (DE); Matthias Wasner, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/436,681

(22) Filed: May 12, 2003

Related U.S. Application Data
(63) Continuation of application No. PCT/EP01/12603, filed on Oct. 31, 2001.

(30) Foreign Application Priority Data
Nov. 11, 2000 (DE) 100 56 012.1

(51) Int. Cl. A61F 13/00; A61F 15/00

(52) U.S. Cl. 602/41

(57) ABSTRACT

A self-adhesive backing assembly for medical use having a backing to which an adhesive coating is applied. In one embodiment of the invention, the backing assembly includes an aluminum layer between the backing and the adhesive coating.

### CROSS-REFERENCE TO RELATED APPLICATIONS

This is a continuation application of PCT/EP01/12603, filed Oct. 31, 2001, which is incorporated herein by reference in its entirety, and also claims the benefit of German Priority Application No. 100 56 012.1, filed Nov. 11, 2000.

### FIELD OF THE INVENTION

The invention relates to a self-adhesive backing material for medical use with a backing to which an adhesive coating is applied.

### BACKGROUND OF THE INVENTION

Transdermal therapeutic systems (TTS) for delivering active substances through the skin have been known for a long time. The topical application of drugs by way of active substance patch systems offers two main advantages: First, this form of administration produces first-order release kinetics of the active substance, thereby enabling a constant level of active substance to be maintained in the body over a very long period. Secondly, the path of uptake through the skin avoids the gastrointestinal tract and also the first liver passage. As a result, selected drugs may be effectively administered in a low dose. This is particularly advantageous when the drug is desired to act locally while avoiding a systemic effect. This is the case, for example, with the treatment of rheumatic joint complaints or muscular inflammation.

One embodiment of such transdermal systems which has been well described in the technical literature is that of matrix systems or monolithic systems, in which the drug is incorporated directly into the pressure sensitive adhesive. In the ready-to-apply product, a pressure sensitively adhesive matrix comprising active substance of this kind is equipped on one side with a backing impermeable to the active substance, while on the opposite side there is a backing film equipped with a release layer, which is removed prior to application to the skin (kleben&dichten, No. 42, 1998, pp. 26 to 30).

Highly specific requirements are imposed on this backing film of a transdermal therapeutic system:

Depending on the size of the patch applied the material used must preferably posses sufficient flexibility and elasticity to ensure adequate patient comfort.

If the backing film used is too rigid the patient has the unpleasant perception of an alien body. Additionally, when applied to moving areas of the body, an insufficiently elastic backing material may result in detachment of parts of the product or even of the entire product. That would prevent the transport of active substance through the skin and jeopardize the effectiveness of the TTS.

On the other hand, the backing layer is preferably configured to reliably prevent loss of active substance over the period of storage. The period of storage is the time between manufacture of the product and its application to the patient. The maximum time frame is frequently defined by way of the maximum shelf life, which generally encompasses three years. From this long period of time it is clear that the material used preferably constitutes a very effective barrier both to the active substance used and to the auxiliaries employed.

The problem surrounding these two requirements is in particular that existing barrier materials are of poor flexibility and elasticity. Known flexible and elastic backing materials, on the other hand, are generally characterized by a very low barrier effect with respect to migratable molecules.

Backing materials for bandage systems play an important part in particular in wound care. In these applications the focus is on the wear comfort of the patient, the duty of care applying in particular to injuries to high-movement joints such as in the knee and elbow regions, for example, or on the hand. The materials employed in this context have in the past frequently been very soft PVC films, which have slowly been replaced by polyolefin films. Modern products are frequently equipped with a nonwoven backing.

EP 0 749 756 A2 describes for example a nonwoven material based on polyester elastomers as a backing material for a bandage for wound care. Owing to the excellent elasticity and conformability of this material a high degree of wear comfort is achieved. It is further increased by the high water vapor permeability of the backing described.

Nonwovens, however, have a microporous structure which contradicts an effective barrier action. Migratable ingredients of a patch system can be volatilized very rapidly through such materials. For this reason such a system is unsuitable for use in the field of active substance patches.

A very good barrier effect is possessed in particular by films of polyethylene terephthalate (PET). Consequently, this material is widespread in the packing industry in the sector of flavor tight and gas tight packaging. In the area of Transdermal Therapeutic Systems as well PET is employed with great frequency. Virtually all of the release films used here consist of PET.

The reason for this lies not only in the outstanding barrier effect of PET but also in its extreme mechanical stability. This leads to distinct advantages during the production of these systems, here in particular in the course of coating and converting, by punching for example. Flexible materials are very much more difficult to deal with in these operating steps.

As a backing material, PET is unsuitable owing to its low flexibility and elasticity, despite being very widespread as such in the absence of suitable alternatives. Another reason for this is that conventional active substance patches can be kept very small in terms of their dimensions. In the case of drugs with a systemic action, moreover, the site of dermal application is relatively unimportant, and so the patch can be applied in the area of body regions where there is very little movement. The chest area in particular may be mentioned here.

The described problem of flexible backing materials having good barrier properties is known in the literature, and numerous solutions are given.

DE 195 46 024 A1 describes for example precisely this difficulty. The disadvantage of a deficient barrier effect on the part of flexible backing films is profitably exploited here. Thus a backing system which is permeable to the active substance is praised as an additional active substance reservoir, allowing a substantial reduction in the overall thickness of the patch system. The reduced thickness achieved in this way raises the flexibility of the overall product and so enhances the desired patient comfort. However, the problem of the loss of active substance which occurs remains unresolved.

A compromise between wear comfort and ease of processing is described by WO 99/12529. There, a unidirectionally elastic backing is used. As a result it is possible to use a material which is rigid in the processing direction, producing substantial advantages during the manufacturing operation. Because of the elasticity perpendicular to this direction, however, an acceptable wear comfort is achieved. The problem of the barrier effect, however, is not mentioned in that text.

A very supple patch is described by WO 98/29143. In that case a backing material is employed which following application to the skin is removed. In the cited document the backing material to be removed is referred to as a “supporting layer”. The underlying pressure-sensitively adhesive layer is given an anti-adhesive finish in order to prevent sticking to the clothing. Achieved as a result is an extremely thin and therefore highly flexible product construction.

Distinct disadvantages of this design, however, exist in respect of the ease or processing of such a system and also the ease of handling for the patient. The absence of a backing during application makes it significantly more difficult to detach the patch following use. Adhesives used generally do not possess the necessary cohesiveness to allow the bandage to be removed in one piece after the period of wear. Additionally, in the course of wear the patch is highly sensitive to mechanical stresses, for example the friction which occurs with the clothing. Moreover, no solution is provided as to how the necessary supporting film adheres to a layer that is configured not to be adhesive.

### SUMMARY OF THE INVENTION

It is an object of the invention to provide a backing material whose backing exhibits a sufficient barrier effect and which avoids the disadvantages known from the prior art. The material ought to be able to be manufactured inexpensively and ought to be ecologically unobjectionable, while also offering pleasant wear comfort in use.

### DETAILED DESCRIPTION OF THE PREFERRED EMBODIMENTS

Proposed in accordance with one embodiment of the invention is a self-adhesive backing material for medical use with a backing to which an adhesive coating is applied, said backing bearing an aluminum layer located between backing and self-adhesive coating.

In a first preferred embodiment of the invention, the aluminum layer has an optical density of more than 1.4, in particular between 2.5 and 3.0.

In a further preferred embodiment of the invention, the backing used comprises polymer films, nonwovens, woven fabrics, and combinations thereof. Backing materials available for selection include polymers such as polyethylene, polypropylene, and polyurethane or else natural fibers. By way of example a metallocene polyethylene nonwoven is suitable.

The metallocene polyethylene nonwoven preferably has the following properties:

a basis weight of from 40 to 200 g/m², in particular from 60 to 120 g/m², and/or

a thickness of from 0.1 to 0.6 mm, in particular from 0.2 to 0.5 mm, and/or

a machine-direction ultimate tensile stress elongation of from 400 to 700% and/or

a cross-direction ultimate tensile stress elongation of from 250 to 550%.

The fibers of the metallocene polyethylene nonwoven preferably have a diameter of from 1 to 50 μm, in particular from 3 to 25 μm.

In addition it has proven advantageous if the metallocene polyethylene nonwoven is characterized by

a force at 25% elongation in the cross direction of from 0.7 to 4 N/cm and/or

a force at 50% elongation in the cross direction of from 0.85 to 6.0 N/cm and/or

a force at 100% elongation in the cross direction of from 1.2 to 8.0 N/cm and/or

a plastic deformation after 5-fold elongation and release by 50% of from 5 to 35%.

In one advantageous embodiment the polymer used is a copolymer of ethylene and an α-olefin having a carbon number from C₄ to C₁₀, the polyolefin possibly having a melt index of between 1 and 20 g/(10 min) and a density of from 860 to 900 kg/m³. The reverse of the metallocene polyethylene nonwoven may also have been given an anti-adhesive treatment.

For the adhesive coating it is preferred to use commercially customary pressure sensitive adhesives based on acrylate or rubber.

The application weight of the adhesive on the backing lies in particular in the range from 100 to 500 g/m², more preferably 300 g/m².

In another preferred version of the invention the adhesive is composed of a pressure-sensitively adhesive matrix containing active substances if desired.

By way of example the matrix can be free from mineral oils and may comprise the following constituents:

a) synthetic framework polymers based on polyisobutylene at from 25 to 90% by weight,

b) tackifier resins at from 5 to 40% by weight,

c) at least one insoluble hydrophilic filler having an average particle size of less than 100 μm at from 10 to 60% by weight, and

d) if desired, a drug at from 0.001 to 20% by weight.

In a first advantageous embodiment the polyisobutylene is composed of high molecular mass PIB at from 5 to 30% by weight and low molecular mass PIB at from 20 to 60% by weight.

A typical pressure sensitive adhesive of the invention is therefore composed of the following components:

| | | |
|---|---|---|
| High molecular mass PIB | 5-30% | preferably |
| | by weight | 10-20% by weight |
| Low molecular mass PIB | 20-60% | preferably |
| | by weight | 30-50% by weight |
| Tackifier resin | 5-30% | preferably |
| | by weight | 5-20% by weight |
| Hydrophilic filler | 20-60% | preferably |
| | by weight | 30-50% by weight |
| Optional drug | 0.001-20% | preferably |
| | by weight | 1.0-5.0% by weight |

As an option it is also possible to add up to 20% by weight of a permeation promoter.

The stated formula ingredients are defined more precisely as follows:

High molecular mass PIB:

Polyisobutylene having a weight-average molecular weight (M_{w}) of from 500,000 to 1,100,000, preferably between 50,000 and 850,000. Such polymers are available commercially for example under the trade names Oppanol B100 (BASF) or Vistanex MM-L80 (Exxon).

Low molecular mass PIB:

Polyisobutylene having a weight-average molecular weight (M_{w}) of from 40,000 to 120,000, preferably between 60,000 and 100,000. Such polymers are available commercially for example under the trade names Oppanol B15 (BASF) or Vistanex LMMH (Exxon).

Tackifier resins:

Tackifier resins comprising partly or fully hydrogenated hydrocarbons and also esters or terpenes having weight-average molecular weights (M_{w}) of between 270 and 1200. Such tackifier resins are available commercially for example under the trade names Escorez® (Exxon), Wingtak® (Goodyear), and Regalite® (Hercules).

Amorphous poly-α-olefin:

Amorphous copolymers based on ethylene and propylene, butylene or 1-hexene. The preferred weight-average molecular weight (M_{w}) is from 5,000 to 100,000, more preferably between 10,000 and 30,000. Such polymers are available commercially for example under the trade names Eastoflex® (Eastman) or Vestoplast® (Hüls).

Hydrophilic filler:

Hydrophilic particles insoluble in the stated polymer matrix and based on cellulose. Preference is given to an average particle size of less than or equal to 100 μm with as uniform as possible a surface. Such materials are available commercially for example under the trade names Avicel (FMC) and Elcema (Degussa-Hüls).

Preparation takes place preferably in a process in which all of the components are homogenized in the melt with no solvent being added. Particular preference is given to processing all of the components in a continuous or batch wise operation at a temperature below 100° C.

The adhesive is distinguished by outstanding adhesion properties to the skin, by easy and painless redetachability, and in particular by its extremely low potential to induce skin irritation. The preparation operation proceeds with the complete omission of solvents.

Without making any claim to completeness, typical active substances in the adhesive in the context of the present invention include the following:

| Indication: | Active substance |
|---|---|
| Antimycotics | naftifine |
| | amorolfine |
| | tolnaftate |
| | ciclopirox |
| Antiseptic | thymol |
| | eugenol |
| | triclosan |
| | hexachlorophene |
| | benzalkonium chloride |
| | clioquinol |
| | quinolinol |
| | undecenoic acid |
| | ethacridine |
| | chlorhexidine |
| | hexetidine |
| | dodicine |
| | iodine |
| Nonsteroidal | glycol salicylate |
| antirheumatics | flufenaminic acid |
| | ibuprofen |
| | etofenamat |
| | ketoprofen |
| | piroxicam |
| | indomethacin |
| Antipruritics | polidocanol |
| | isoprenaline |
| | crotamiton |
| Local anesthetics | benzocaine |
| Antipsoriatics | ammonium bitumasulfonate |
| Keratolytics | urea |
| | salicylic acid |

Mention may also be made of hyperemic active substances such as natural active substances of Cayenne pepper or synthetic active substances such as nonivamide, nicotinic acid derivatives, preferably benzyl nicotinate or propyl nicotinate.

If desired, the open, adhesive side of the backing material, the side to be applied to the skin, can be lined with a redetachable protective liner layer. It is additionally possible to dispose a customary wound contact material on the self-adhesive coating.

With particular advantage the backing material, with or without wound contact material, can be punched into the shape of patches or bandages, allowing specific covering of wounds and/or controlled delivery of active substances to the skin.

The backing of the backing material for medical use is notable in particular for the fact that on one side it is provided with a barrier layer which is impervious to gases, water vapor, drugs, and aroma substances. Another feature of the backing beside its effective barrier properties is its effective flexibility.

The present invention thus describes the provision of backings in particular for transdermal therapeutic systems (TTS) with a barrier layer of aluminum. This aluminum layer is generated in one preferred embodiment by vapor deposition of the metal onto the film under a high vacuum.

The advantage of a barrier layer of aluminum is that on the one hand the metal is not toxic and on the other hand, through passivation of the metal surface, the barrier layer is made highly resistant to attack by the ingredients of the TTS.

A further advantage of the aluminum layer applied by vapor deposition is that there is little effect on the mechanical properties of the polymer films.

Flexibility and surface structure of the films alter only to a minor extent. As a consequence it is even possible to use embossed films as backings for vapor deposition, without permanently affecting the structure of the embossing. This property of the barrier layer is particularly remarkable in view of effective anchoring of adhesive on the backing. In accordance with one embodiment of the invention the backing material for medical use ought to have a high elasticity either with or without any aluminum layer.

A further measure of the nature of the barrier layer, besides the parameters of water vapor permeability and oxygen permeability, is the optical density. For customary barrier layers optical densities of 1.4 and above are employed. Preference is given to an optimum comprising an optical density of between 2.5 and 3.0, allowing a reduction in permeability by a factor of up to 100. At optical densities >3.0 the barrier effect reaches saturation and at the same time the anchoring of the aluminum to the film goes down.

For the working of the films a corona pretreatment is advisable, since the vapor deposition requires a surface tension of at least 38 dynes.

Surface treatment is effected in accordance with the standard industrial techniques. At the end of the manufacturing operation the film is guided over a grounded, bright aluminum or steel roll. Located above the roll is an insulated electrode, resulting in continuous, automatic discharge which impinges on the film surface. A high-frequency generator generates alternating voltages of from 10 to 20kV with frequencies of between 10 to 60 kHz (J. Nentwig, “Lexikon Folientechnik, VCH Weinheim (1991), pp. 80-82). The surface tension of the film should be checked prior to vapor deposition and corrected where appropriate by further corona treatment, since it goes down as the period of storage increases.

Where necessary the mechanical stability of the vapor-deposited aluminum layer can be improved by means of two additional measures. First, the adhesive containing active substance can be laminated directly to the barrier layer; secondly, there is in an increase in the mechanical stability by application of a primer layer or a protective coating material to the vapor-deposited aluminum.

In one typical application a metallocene PE film having a thickness of 85 μm is coated with aluminum by vapor deposition. The optical density is 1.47.

As a function of the optical density the thickness of the vapor-deposited barrier layer is in the range from 300 to 400 Å, the optical density normally being employed to describe the aluminum layer. These thin layers ensure that the mechanical properties of the film are affected only to a very slight extent. This can be demonstrated by means of hysteresis measurements on the corresponding films.

Hysteresis

| | Film without barrier layer | Film with barrier layer |
|---|---|---|
| εₑₗ | 43.63 | 43.40 |
| εₚₗₐₛₜ | 6.37 | 6.60 |

A further advantage of coating the base materials by vapor deposition is that the aluminum layer forms an impervious barrier layer which exhibits ideal conformation to the structural qualities of the base material, laminates comprising aluminum foils. In the case of a laminate comprising a base film and an aluminum foil the inflexibility of the aluminum foil is transferred to the base material, which loses its flexibility as a result.

Water vapor permeability

| Temperature | Uncoated film | Coated film |
|---|---|---|
| [° C.] | [g/m²/d] | [g/m²/d] |
| 27 | 3.46 | 2.82 |
| 27 | 3.32 | 2.74 |
| mean | 3.39 | 2.78 |
| 37.8 | 7.82 | 6.18 |
| 37.8 | 7.54 | 6.10 |
| mean | 7.68 | 6.14 |

In this case the barrier properties of the film coated by vapor deposition with respect to water vapor can be improved by approximately 20%, such an improvement already being enough for use as a backing for TTS with low active substance concentrations.

The oxygen permeability films is measured using an OX-TRAN 100 instrument. For the measurement a section of film is used which has an area of 100 cm², together with a 5 cm² mask. The film coated by vapor deposition exhibits a barrier effect toward oxygen which is improved by 39% as compared with the base film. The individual results are listed in the table below:

Oxygen permaebility

| | Temperature/rel. humidity | | |
|---|---|---|---|
| Sample | [° C.]/[%] | Uncoated film | Coated film |
| Cell A | 37.8/40 | 10385 | 6050 |
| Cell B | 37.8/40 | 9310 | 5917 |
| Mean | | 9847.5 | 5983.5 |

The barrier effect with respective to active substance is determined by penetration measurements in a VanKel enhancer cell. A patch doped with ibuprofen at 5% by weight is bonded to the coated side (barrier layer) of the film. The uncoated side is placed in contact with a phosphate buffer. After seven days the phosphate buffer is removed and analyzed by HPLC. In this case the barrier effect for the active substance ibuprofen is increased by 30%.

| Measurement | | |
|---|---|---|
| period | Uncoated film | Coated film |
| [d] | [μg/cm²] | [μg/cm²] |
| 7 | 164 | 118 |

Particularly advantageous backing material for medical use, according to the invention, is described below with reference to a number of examples, without wishing thereby to subject the invention to any unnecessary restriction.

### EXAMPLE 1

A pressure sensitive adhesive composed of 90% by weight SEBS and 5% by weight lauroglycol and doped with 5% by weight ibuprofen is coated onto the backing material through a slot die. The active substance composition laminated onto the backing material is then rolled onto the backing material together with a polyester release film under pressure between two press rolls for the final anchoring of the composition.

### EXAMPLE 2

A pressure sensitive adhesive composed of 51.7% by weight Vistanex LM MH, 27.3% by weight Vistanex MM L80, and 16.0% by weight Escorez 5690 and doped with 5% by weight ibuprofen is coated onto the backing material through a slot die. The active substance composition laminated onto the backing material is then rolled onto the backing material together with a polyester release film under pressure between two press rolls for the final anchoring of the composition.

### EXAMPLE 3

A pressure sensitive adhesive composed of 52.7% by weight Vistanex LM MH, 27.3% by weight Vistanex MM L80, and 18.0% by weight Escorez 5690 and doped with 2% by weight ibuprofen is coated onto the backing material through a slot die. The active substance composition laminated onto the backing material is then rolled onto the backing material together with a polyester release film under pressure between two press rolls for the final anchoring of the composition.

### EXAMPLE 4

A pressure sensitive adhesive composed of 50.8% by weight Vistanex LM MH, 25.9% by weight Vistanex MM L80, 17.3% by weight Escorez 5690, and 5.0% by weight zinc oxide and doped with 1% by weight indomethacin is coated onto the backing material through a slot die. The active substance composition laminated onto the backing material is then rolled onto the backing material together with a polyester release film under pressure between two press rolls for the final anchoring of the composition.

What I claim is:

1. A self-adhesive backing assembly for medical use comprising:
a backing;
an adhesive disposed adjacent said backing; and
an aluminum layer disposed between said backing and said adhesive.

2. The backing assembly of claim 1, wherein said aluminum layer has an optical density greater than 1.4.

3. The backing assembly of claim 1, wherein said aluminum layer has an optical density that is between 2.5 and 3.0.

4. The backing assembly of claim 1, wherein said backing comprises at least one polymer film.

5. The backing assembly of claim 4, wherein said polymer film is selecting from a group consisting of: polyethylene, polypropylene and polyurethane.

6. The backing assembly of claim 1, wherein said backing comprises at least one nonwoven.

7. The backing assembly of claim 1, wherein said backing comprises at least one nonwoven laminate.

8. The backing assembly of claim 1, wherein said backing comprises a metallocene-polyethylene nonwoven.

9. The backing assembly of claim 8, wherein said metallocene-polyethylene nonwoven has a basis weight that is between 40 to 200 g/m².

10. The backing assembly of claim 8, wherein said metallocene-polyethylene nonwoven has a basis weight that is between 60 to 120 g/m².

11. The backing assembly of claim 8, wherein said metallocene-polyethylene nonwoven has a thickness of between 0.1 to 0.6 mm.

12. The backing assembly of claim 8, wherein said metallocene-polyethylene nonwoven has a thickness of between 0.2 to 0.5 mm.

13. The backing assembly of claim 1, wherein said adhesive comprises:
a) one or more synthetic framework polymers based on polyisobutylene at from 25 to 90% by weight,
b) one or more tackifier resins at from 5 to 40% by weight, and
c) at least one insoluble hydrophilic filler having an average particle size of less than 100 μm at from 10 to 60% by weight.

14. The backing assembly of claim 13, wherein said adhesive is free from mineral oils.

15. The backing assembly of claim 13, wherein said adhesive further comprises a drug at from 0.001 to 20% by weight.

16. The backing assembly of claim 1, wherein said aluminum layer is coated with a protective layer to protect against mechanical stresses.

17. The backing assembly of claim 16, wherein said protective layer is applied to said aluminum layer under a high vacuum.

18. A method of assembling a self-adhesive backing assembly for medical use, said method comprising:
providing a backing;
providing an adhesive adjacent said backing; and
applying an aluminum layer to said backing by vapor deposition under a high vacuum so that said aluminum layer is between said backing and said adhesive.
